(19) United States
(12) Patent Application Publication
(10) Pub. No.: US 2026/0108562 A1
(43) Pub. Date: Apr. 23, 2026

(54) Method for Preparing Targeted Dermal Papilla Cell-Derived Exosomes, Exosome Product, Liquid Hair Growth Formulation, and Method of Using Liquid Hair Growth Formulation

(71) Applicant: Chun-Chou Tsai, Taipei (TW)

(72) Inventor: Chun-Chou Tsai, Taipei (TW)

(21) Appl. No.: 19/352,402

(22) Filed: Oct. 7, 2025

Related U.S. Application Data
(60) Provisional application No. 63/710,017, filed on Oct. 21, 2024.

(30) Foreign Application Priority Data
Sep. 5, 2025 (TW) 114134126

(51) Int. Cl. A61K 35/36; A61P 17/14; C12N 5/071

(52) U.S. Cl. CPC A61K 35/36; A61P 17/14; C12N 5/0627

(57) ABSTRACT

A method for preparing targeted dermal papilla cell-derived exosomes is provided. The method involves first isolating a plurality of dermal papilla cells (DPCs) with a cell surface marker CD133 from the hair follicles of a healthy donor, in a xeno-free and serum-free environment in GTP lab in Taiwan (or GMP lab in international). Subsequently, a cell culture medium containing a plurality of exosomes is collected, wherein these exosomes possess surface markers such as CD9, CD63, and CD81. Finally, the exosomes are isolated, concentrated, and purified from the culture medium using techniques such as tangential flow filtration system (TFF system) or size exclusion chromatography, thereby obtaining the final targeted exosome lyophilized product. A liquid hair growth formulation formulated with this exosome product is also provided.

### CROSS-REFERENCE TO RELATED APPLICATION

This application claims priority to U.S. Provisional Patent Application No. 63/710,017, filed on Oct. 21, 2024, and Taiwanese Patent Application No. 114134126, filed on Sep. 5, 2025. The entire contents of both applications are incorporated herein by reference.

### BACKGROUND

### Technical Field

The present invention relates to a targeted exosome for hair regeneration, and more particularly to a method for preparing a targeted exosome for hair regeneration and a preparation thereof.

### Description of Related Art

Hair loss, especially androgenetic alopecia (AGA), is a widespread condition. The main pathological mechanism of AGA is associated with the increased sensitivity of dermal papilla cells (DPCs) in hair follicles to androgens. In patients affected by AGA, DPCs exhibit elevated expression of 5α-reductase and androgen receptor (AR), which shortens the anagen phase of hair follicles and results in hair loss. Conventional clinical treatments mainly include topical hair growth products (such as Minoxidil) and oral medications (such as Finasteride or Dutasteride). The mechanism of action of minoxidil is vasodilation, which enhances blood supply of hair follicles. Oral medications decrease androgen effects by inhibiting 5α-reductase. However, these treatments usually have significant disadvantages, such as edema and sexual dysfunction, and require long-term continuous use. Upon discontinuation, hair loss tends to resume. Therefore, both academia and industry are continuously seeking more effective hair regeneration technologies with fewer adverse effects.

Clinical classification of AGA forms the basis for assessing severity and evaluating therapeutic outcomes. Male pattern hair loss (MPHL) is typically classified using the Norwood-Hamilton scale, ranging from Stage I (no significant hair loss) to Stage VII (only little hair remains at the occipital and temporal regions), characterized by a receding hairline and thinning hair on the vertex. Female pattern hair loss (FPHL) is classified using the Ludwig scale, which ranges from Stage I to Stage II. Although both male and female pattern hair loss exhibit common features such as follicular miniaturization and deregulation of hair cycle dynamics, certain studies consider them distinct entities with different pathological mechanisms.

In recent years, exosomes have attracted widespread attention as the “next-generation acellular therapy.” Exosomes possess the potential to act as intercellular messengers and are considered to play a key role in tissue regeneration. Recent studies have indicated that exosome therapy shows potential benefits in treating androgenetic alopecia. Exosome therapy primarily promotes hair regeneration through mechanisms such as activating dermal papilla cells, modulating inflammation, and stimulating angiogenesis. In current clinical studies, exosomes are primarily derived from adipose-derived stem cells (ADSCs) or hair follicle stem cells (HFSCs). The main administration methods involve topical application combined with microneedling or injection of autologous micrograft suspensions.

Several studies have provided preliminary evidence supporting the efficacy of exosome therapy. For example, a retrospective study of 39 male pattern hair loss patients demonstrated that patients treated with microneedling (invasive) in combination with exosomes derived from adipose-derived stem cells significantly increased hair density and thickness after 12 weeks (3 months). Another prospective study demonstrated that patients treated with microneedling (invasive) combined with exosomes experienced an average increase of 35 hairs per cm² in hair density after 12 months. Additionally, the results showed that the side effects were generally mild and short-lived, including slight redness or itching, with no serious adverse events reported, indicating a favorable safety profile.

Although preliminary results are encouraging, existing studies still have certain limitations. For example, it remains unclear which exosomes can effectively promote the catagen (regression) phase of the hair follicles while delay anagen (growth) phase of the hair follicles, thereby promoting hair regenerated, to increase regional hair density and hair root thickness, and which administration method can provide greater convenience for patients with androgenetic alopecia. Furthermore, more in-depth investigations are required to elucidate the molecular mechanisms underlying the regenerative effects of exosomes, particularly their ability to activate key signaling pathways such as Wnt/β-catenin, with the goal of developing more standardized, controllable, and consistently effective preparation methods.

Accordingly, a key challenge in current technologies is to develop a targeted exosome-based hair growth formulation capable of efficient cell source selection, optimized preparation procedures, and consistent production of exosomes exhibiting superior efficacy, safety, and transdermal penetration.

### SUMMARY

The present invention aims to solve the foregoing problems by providing a unique “super-donor” screening strategy and a precise manufacturing process. The resulting exosomes have been proven to effectively promote hair growth and enhance the health and vitality of hair follicle cells at the molecular level, bringing revolutionary progress to hair regeneration therapies.

An aspect of the present invention is to provide a method for preparing targeted dermal papilla cell-derived exosomes, the method comprising the following steps: isolating a plurality of dermal papilla cells expressing a cell marker CD133 (hereinafter referred to as CD133+ DPCs); culturing the CD133+ DPCs in a two-dimensional culture for primary culture, thereby obtaining a plurality of cell masses by the growth of the CD133+ DPCs; subculturing the cell masses to obtain a plurality of spindle-shaped cell lineages exhibiting adherent growth; collecting a cell culture medium containing a plurality of exosomes present in a surrounding environment during growth of the cell lineages; isolating the exosomes from the cell culture medium to obtain a high-concentration and high-purity exosome solution, wherein the exosomes express surface markers such as CD9, CD63, and CD81.

According to an embodiment of the present invention, the dermal papilla cells are obtained from the occipital hair follicles of a healthy human donor conforming to local health regulations, wherein the healthy human donor is aged 35 to 45 years, and the gender is not limited.

According to an embodiment of the present invention, the conditions of the primary culture and the subculture comprise being carried out in a laboratory of a Taiwan GTP (or GMP Lab in international) cell preparation facility, being conducted under a xeno-free and serum-free culture environment, and maintaining a temperature of 37° C. and a carbon dioxide concentration of 5%.

According to an embodiment of the present invention, the methods for isolating the CD133+ DPCs comprise an immunomagnetic bead separation method.

According to an embodiment of the present invention, the methods for isolating the exosomes comprise a tangential flow filtration system, a size exclusion chromatography, or a combination thereof.

According to an embodiment of the present invention, the composition of the exosome solution comprises the exosomes at a concentration of 3.5×10⁸/mL to 4.5×10¹⁴/mL and proteins at a concentration of 3000 to 3200 mg/mL, wherein the exosomes have a particle size of 30 to 160 nm.

In another aspect, the present invention provides an exosome product. A method for preparing the exosome product comprises the following steps: obtaining the exosome solution by using the above-described targeted exosome preparation method; and lyophilizing the exosome solution to obtain the exosome product in a lyophilized powder form, wherein a positivity rate for a surface marker CD9 is greater than 20%, for a surface marker CD63 is greater than 18%, and for a surface marker CD81 is greater than 7%.

In another aspect, the present invention provides a liquid hair growth formulation prepared by dispersing the above-described exosome product in a solvent. The liquid hair growth formulation comprises the exosomes at a concentration of 10³ to 10⁸/mL.

According to an embodiment of the present invention, the solvent of the liquid hair growth formulation is normal saline or a solution comprising other components in liquid form.

In another aspect, the present invention provides a method of using the above-described liquid hair growth formulation, the method comprising the following steps: spraying the liquid hair growth formulation onto a target hair growth site on the scalp of an individual; and massaging the target hair growth site of the individual to promote penetration of the exosomes into a plurality of hair follicles of the scalp, thereby stimulating the hair follicles to grow hair.

The present invention discloses an innovative targeted exosome-based liquid hair growth formulation and preparation method thereof, with the core advantage of obtaining exosomes through a precise culture and purification process from hair follicle dermal cells of strictly selected “super donors.” The exosomes have been proven effective in promoting hair follicle cell proliferation and activating growth-related signaling pathways in in vitro tests. Moreover, significant efficacy has been demonstrated in volunteer cases, including promoting hair growth/density and extending hair follicle telomere length. The liquid hair growth formulation possesses excellent transdermal permeability and safety, providing a revolutionary solution for hair regeneration therapy.

The foregoing summary is provided to present a simplified overview of the invention so as to facilitate a basic understanding of the invention. The summary is not intended to be an exhaustive disclosure of the invention, nor is it intended to identify key or critical elements of the embodiments or to define the scope of the invention. Upon reading the following detailed description, those skilled in the art will readily understand the fundamental spirit of the invention, other objectives of the invention, and the technical means and implementations adopted herein.

### BRIEF DESCRIPTION OF THE DRAWINGS

FIG. 1 is a diagram showing morphological changes of hair follicle dermal papilla cells isolated from hair follicle tissue at different culture stages, wherein (a) and (b) respectively show images of the hair follicle dermal papilla cells derived from Donor 1 and Donor 2.

FIG. 2A-2D are comparative diagrams showing before and after use conditions of four volunteers from a group of 500 volunteers.

FIG. 3 is a diagram showing correlation between age and number of days required for hair regrowth.

### DETAILED DESCRIPTION

According to the foregoing, the present invention provides a novel and non-invasive method for preparing exosomes derived from dermal papilla cells (DPCs) of hair follicles and applying the same to the treatment of androgenetic alopecia, alopecia areata, and other fields of hair regeneration. The core of the present invention lies in obtaining targeted exosomes with superior hair growth efficacy through precise selection and culture procedures, and formulating the exosomes into a safe and effective liquid hair growth formulation

In order to fully describe the embodiments of the present invention, explanatory descriptions of different aspects and specific embodiments are provided below. These are not limited to any particular form of implementation or application, but rather encompass features and methodological steps of multiple specific embodiments. Different embodiments may achieve the same or equivalent functions and steps, thereby demonstrating the flexibility of the present invention.

### Source and Acquisition of DPCs of Hair Follicles.

The key of the present invention lies in obtaining the high-quality DPCs of the hair follicles from healthy donors meeting specific criteria so as to ensure that the exosomes subsequently prepared possess optimal activity.

The conditions for screening the healthy donors comprise meeting a relevant donor criterion stipulated by applicable local health regulations. For example, in Taiwan, the donors are required to meet donor criterion stipulated by the Ministry of Health and Welfare. The donor criterion in Taiwan comprises that tests for HIV I/II, syphilis, HBV, HCV, CMV, and other items must all be negative in a health examination conducted within two weeks prior to donation. According to one embodiment of the present invention, a gender of the healthy donors is not limited (male or female). According to another embodiment of the present invention, a collection site of the DPCs of the hair follicles is an occipital region of the healthy donors.

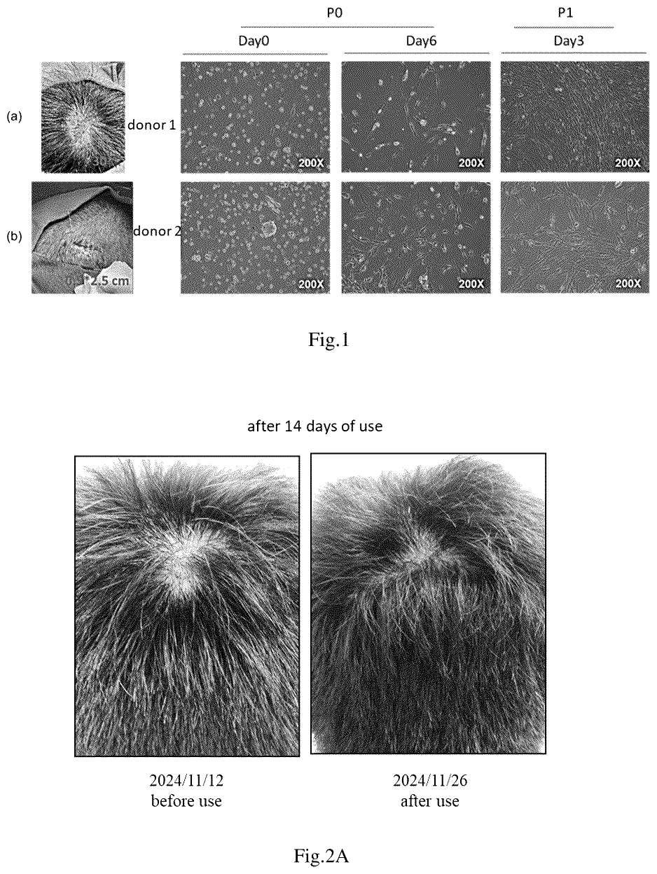

According to another embodiment of the present invention, a collection of the DPCs of the hair follicles may be performed surgically to obtain hair follicle tissue from the skin. Please refer to a left image of FIG. 1, for example, punch biopsy or micro-puncture techniques similar to hair transplantation may be used to collect approximately 30 to 50 hair follicles from scalp tissues of a healthy donor bearing hair follicular units. This method of harvesting the DPCs of the hair follicles may leave small reddish punctate wounds on the scalp.

As shown in the left image in (b) of FIG. 1, another method for collecting the DPCs of the hair follicles involves a microdissection technique. With the aid of a microscope, skin tissue of approximately 0.5×2.5 cm² is excised from the occipital scalp of a healthy donor, and multiple intact hair follicle tissues are precisely dissected therefrom. However, this collection method may leave a wound scar of about 0.5×2.5 cm² on the scalp.

After obtaining the DPCs of the hair follicles from the occipital regions of several healthy donors, individuals exhibiting the most significant performance in terms of cell proliferation capacity, exosome production, or exosome activity (such as promoting hair follicle cell proliferation or hair growth) will be selected. In subsequent experiments, the selected individuals will serve as super donors of the DPCs of the hair follicles.

In vitro Culture of the DPCs of Hair Follicles and Isolation of Exosomes.

In this embodiment, the DPCs of the hair follicles obtained from the super donors in Taiwan are subjected to screening and culture in a cell preparation facility compliant with Good Tissue Practice (GTP) standards of Taiwan.

First, the screening process employs an immunomagnetic bead separation method, such as MACS MicroBead, to obtain the DPCs of the hair follicles carrying a cell marker CD133 (hereinafter referred to as CD133+ DPCs). Screening results are shown in Table 1. Data in Table 1 indicate that an isolation rate of CD133+ DPCs is approximately 3.9×10⁻³, which is consistent with findings reported in the international literature.

TABLE 1
Quantity of the DPCs of the hair follicles
with or without the cell marker CD133.

| Presence of cell marker CD133 | Cell quantity |
|---|---|
| Present | 2.4 × 10⁵ |
| Absent | 6.1 × 10⁷ |

Next, cell cultures of the DPCs of the hair follicles are performed. Both a primary culture and a subculture are conducted under a xeno-free and serum-free culture environment for expansion of the cell quantity. The cell cultures are maintained at a temperature of 37° C. and a carbon dioxide concentration of 5%.

First, the purified CD133+ DPCs are subjected to the primary culture (hereinafter referred to as P0). Please refer to microscopic images labeled P0 in a middle portion of FIG. 1. At the P0 stage, the CD133+ DPCs appear as cell masses on day 0, whereas on day 6 the CD133+ DPCs begin to adhere to the culture dish and gradually elongate into a spindle shape.

Subsequently, the spindle-shaped CD133+ DPCs are subjected to the subculture (hereinafter referred to as P1) to obtain a plurality of spindle-shaped cell lineages grown adherently. Please refer to microscopic images labeled P1 in a right portion of FIG. 1. As shown in the images of day 3 subculture, the CD133+ DPCs are already spindle-shaped and exhibit well growth status, forming a dense spindle-shaped cell layer. When the CD133+ DPCs reach approximately 80% of the culture dish (about 3 days), a culture medium is collected for subsequent exosome preparation.

In a laboratory facility of GTP cell preparation, an exosome solution is isolated and concentrated from the collected cell culture medium by a tangential flow filtration system (TFF system), a size exclusion chromatography (SEC), or a combination of both. Finally, the obtained exosome solution is subjected to a lyophilization process after freezing at −80° C. for 72 hours and then subjected to a freeze-drying process to obtain a final targeted exosome product in lyophilized powder form (hereinafter referred to as exosome product).

### Composition and Characterization Analysis of Exosome Product.

Nanoparticle Tracking Analysis (NTA) is a technique used to analyze characteristics of nanoparticles in a suspension. The NTA works by illuminating a sample with laser light and capturing trajectories of particles in liquid caused by Brownian motion through a microscope equipped with a high-resolution camera. The NTA individually tracks movement of each particle and calculates a hydrodynamic diameter based on Stokes-Einstein equation, thereby obtaining a particle size distribution and concentration. The NTA can accurately measure nanoparticles ranging from approximately 10 to 2000 nm and provides multidimensional information on particle size distribution, concentration, and aggregation state.

According to some embodiments of the present invention, the cell culture medium of the CD133+ DPCs purified by the TFF system typically contains exosomes at a concentration of approximately 3.5×10⁸ to 4.5×10¹⁴/mL, as well as proteins at a concentration of 3000 to 3200 mg/mL. From a particle size distribution curve of the NTA, the exosomes exhibit a particle size distribution ranging from about 30 to 160 nm. For example, the particle size of the exosomes may be 30, 40, 50, 60, 70, 80, 90, 100, 110, 120, 130, 140, 150, or 160 nm.

Table 2 shows some exosome characteristic data before and after purification of the cell culture medium of the CD133+ DPCs using the TFF system. According to the data in Table 2, it can be estimated that the cell culture medium of the CD133+ DPCs purified by the TFF system contains exosomes at a concentration of approximately 3.92×10¹¹/mL, as well as proteins at a concentration of approximately 3190 mg/mL.

TABLE 2
Exosome Characteristics.

| | | Protein | Exosome | |
|---|---|---|---|---|
| | Dilution | concentration | concentration | Particle size |
| Condition | factor | (mg/mL) | (particles/mL) | (nm) |
| Before TFF | 200 | 3.54 | 7.78 × 10¹⁰ | 53.1 ± 3.8 |
| purification | | | | |
| After TFF | 1000 | 3.19 | 3.92 × 10¹¹ | 54.6 ± 2.9 |
| purification | | | | |

In an identification of exosomes, Nanoparticle Flow Cytometry (NanoFCM) is a highly sensitive technique used for multiparameter analysis of nanoscale single particles such as exosomes. One type of analysis is surface marker analysis. The NanoFCM uses antibody staining to analyze the surface markers of exosomes at the single-particle level, such as commonly markers CD9, CD63, and CD81, thereby confirming identity and heterogeneity of the exosomes in the sample. The analysis results are summarized in Table 3.

TABLE 3
Positive rates of surface markers CD9, CD63, and CD81.

| | Number of | | |
|---|---|---|---|
| Surface protein | positive | Number of | Positivity rate |
| marker | particles | negative particles | (%) |
| CD9 | 819 | 2980 | 21.6 |
| CD63 | 737 | 3281 | 18.3 |
| CD81 | 353 | 4195 | 7.8 |

Since CD9, CD63, and CD81 are internationally recognized core surface markers by the International Society for Extracellular Vesicles (ISEV), the results in Table 3 demonstrate that the detection of these surface markers is sufficient to confirm the presence of exosomes in the exosome product purified by TFF.

### Preparation of Liquid Hair Growth Formulation.

The lyophilized exosome product is dispersed in normal saline (or a solution comprising other components in liquid form) to achieve a final working concentration of exosomes of at least 10³ particles/mL, thereby obtaining a liquid hair growth formulation for subsequent experiments. According to some embodiments of the present invention, the exosome product and normal saline are mixed at a weight ratio ranging from 1:99 to 1:249, resulting in a liquid hair growth formulation with an exosome concentration of 10³ to 10⁸ particles/mL (for example, 10³, 10⁴, 10⁵, 10⁶, 10⁷, or 10⁸ particles/mL).

### Skin Penetration Experiment of Exosomes.

In this experimental example, a skin penetration experiment was conducted on the liquid hair growth formulation to verify its potential application as a liquid hair growth formulation.

In the percutaneous permeation evaluation experiment, the transdermal absorption capacity of the liquid hair growth formulation was assessed. The experiment was performed using a Franz diffusion cell (Hanson Phoenix DB-6 Manual Diffusion System) system equipped with Cytiva (10404006) membrane.

The experimental method involved adding 2 mL of the liquid hair growth formulation into a donor chamber and adding 15 mL of phosphate-buffered saline (PBS) into a receiver chamber. Throughout the experiment, the entire skin penetration system was maintained at 32° C. After diffusion for 4 or 8 hours, the solution from the receiver chamber was collected, and 0.5 mL of the solution was taken for quantitative analysis of the exosome quantity using a nanoparticle tracking instrument (NanoSight NS300) of Malvern Panalytical. The penetration rate of the exosomes was calculated by dividing the quantity of exosomes in the receiver chamber by the number of exosomes in the original liquid hair growth formulation. The results are shown in Table 4. Table 4 indicates that a significant proportion of exosomes could penetrate through the membrane, confirming their good percutaneous penetration ability.

TABLE 4
Penetration Rate of Exosomes.

| | Penetration rate of exosomes (%) |
|---|---|
| Diffusion time (h) | (Average ± Standard Deviation, n = 3) |
| 4 | 27.8 ± 3.4 |
| 8 | 23.9 ± 19.8 |

### Safety Testing of Exosomes.

Next, safety tests were conducted on the liquid hair growth formulation, including anti-inflammatory experiments and in vitro skin irritation tests.

### I. Anti-Inflammatory Assay.

In this assay, macrophages were stimulated with lipopolysaccharide (LPS) to induce an inflammatory response, and the concentrations of pro-inflammatory cytokines, such as TNF-α and IL-6, were quantitatively analyzed to evaluate the anti-inflammatory efficacy of the liquid hair growth formulation.

The liquid hair growth formulation of the present invention was added into a macrophage culture medium as an experimental group in this assay. At the same time, two control groups were established, namely a blank control group containing only the culture medium, and a positive control group in which SB203580, known to effectively inhibit inflammatory signal transduction, was added. SB203580 is an inhibitor of p38 branch of a mitogen-activated protein kinase (MAPK) family (hereinafter referred to as p38 MAPK). The p38 MAPK mainly participates in cellular responses to stress and inflammatory signals. When cells are stimulated by inflammatory factors, the p38 MAPK is activated and subsequently promotes the production of pro-inflammatory cytokines such as TNF-α and IL-6. Therefore, the positive control group is used to validate that the inflammatory response model in the assay is effective and serves as a known baseline for inhibition.

The culture method of macrophages was carried out by incubating the cells overnight at a temperature of 37° C. and a CO₂ concentration of 5%. Subsequently, the culture medium was removed, and different concentrations of the liquid hair growth formulation or positive control substance were added. LPS was added to induce inflammation in the macrophages, and the cells were further cultured for 24 hours. Supernatant of the cell culture was collected, and the concentrations of TNF-α and IL-6 in the supernatant of the cell culture were measured using ELISA kits. At the same time, a cell viability of the macrophages was evaluated by an MTT assay. The assay results are shown in Table 5.

TABLE 5
Anti-inflammatory assay results of liquid hair growth formulation, wherein *p-value < 0.05
indicates a statistically significant difference between the experimental and control groups.

| | | | | | Cell viability |
|---|---|---|---|---|---|
| | | | | | (%) |
| | | | | | (Average ± |
| | Additive | | | | Standard |
| | in culture | | | | Deviation, |
| Group | medium | Concentration | TNF-α (%) | IL-6 (%) | n = 3) |
| Experimental | Liquid hair | 1.25% | 97.57 ± 1.50 | 102.68 ± 1.24 | 96.88 ± 2.08 |
| group | growth | 2.50% | 97.96 ± 1.04 | 103.01 ± 2.58 | 96.86 ± 2.72 |
| | formulation | 5% | 97.49 ± 0.53 | 102.01 ± 1.73 | 96.98 ± 2.67 |
| | | 10% | 98.17 ± 0.40 | 101.99 ± 3.72 | 94.57 ± 5.67 |
| | | 20% | 96.17 ± 0.28 | 102.98 ± 2.26 | 84.85 ± 1.31 |
| Positive | SB203580 | 3 μM | *37.38 ± 0.53 | *28.10 ± 0.59 | 82.80 ± 3.72 |
| control group | | | | | |
| Blank | None | None | 100.00 ± 5.16 | 100.00 ± 0.31 | 100.00 ± 2.80 |
| control group | | | | | |

It was demonstrated from the results in Table 5 that, compared with the control groups, no statistically significant differences were observed in the concentrations of TNF-α and IL-6 in the experimental groups treated with the liquid hair growth formulation at concentrations ranging from 1.25% to 20%. Furthermore, within concentrations ranging from 1.25% to 20%, cell viability all remained above 80%, indicating no apparent cytotoxicity. Accordingly, the results confirm that, at the tested concentrations, the liquid hair growth formulation neither significantly affects the levels of pro-inflammatory cytokines involved in the inflammatory response nor exhibits cytotoxicity to the cells.

### II. In Vitro Skin Irritation Test.

In this test, a reconstructed human epidermis model was used to evaluate skin irritation potential of the liquid hair growth formulation in accordance with the OECD Test Guideline 439 (In Vitro Skin Irritation: Reconstructed Human Epidermis Test Method), a non-animal testing method issued by the Organization for Economic Co-operation and Development (OECD), as an alternative to animal testing.

A test model used herein was an EpiDerm™ model (EPI-200-SIT). The EpiDerm™ model utilizes normal human epidermal keratinocytes (NHEK), which are viable cells obtained from healthy human volunteer donors and specially cultured under an air-liquid interface (ALI) to form a highly differentiated three-dimensional reconstructed tissue model that mimics the structure of human epidermis. The EpiDerm™ model comprises a complete multilayer epidermal structure, including a basal layer, spinous layer, granular layer, and stratum corneum. The cells retain viability and proliferative capacity, thereby simulating the physiological structure and function of human skin. The model is widely applicable in cosmetics, safety evaluation, and dermatological drug testing.

An experimental group was the liquid hair growth formulation. A control groups included a negative control group using DPBS (Dulbecco's Phosphate-Buffered Saline) and a positive control group using 5% SDS (Sodium Dodecyl Sulfate). The DPBS is a commonly used buffer solution in cell biology research and is considered non-irritating to cells or tissues; therefore, its test results serve as a baseline for non-irritation to skin. The SDS is a strong detergent known to cause irritation and damage to the skin; therefore, its test results serve as a baseline for skin irritation.

The skin tissue of the EpiDerm™ model was pre-cultured overnight under standard cell culture conditions (temperature of 37±1° C., humidity of 95±5%, CO2 of 5±1%). Subsequently, the skin tissue was topically exposed to the test substance for 60 minutes, then washed to remove the test substance and transferred to fresh culture medium for sequential cultured periods of 24 hours and 18 hours. Finally, an MTT assay was performed and the relative cell viability was calculated relative to the negative control. A relative cell viability below 50% indicates that the test substance has skin irritation potential. The experimental results are shown in Table 6. The principle of the MTT assay is that enzymes in the mitochondria of viable cells, such as succinate dehydrogenase, reduce the yellow tetrazolium compound MTT into purple formazan crystals, the absorbance of which is then quantitatively measured at a wavelength of 570 nm.

TABLE 6
Results of in vitro skin irritation test, wherein *p-
value < 0.05 indicates a statistically significant
difference between the experimental and control groups.

| | | Relative cell viability (%) |
|---|---|---|
| | | (Average ± Standard Deviation, |
| Group | OD₅₇₀ | n = 3) |
| Negative control | 2.291 ± 0.167 | 100.0 ± 7.3 |
| group | | |
| Positive control group | 0.065 ± 0.002 | *2.8 ± 0.1 |
| Experimental group | 2.428 ± 0.072 | 106.0 ± 3.2 |

As shown in Table 6, the cell viability of the experimental group was greater than 50%. Therefore, according to the United Nations Globally Harmonized System of Classification and Labelling of Chemicals (UN GHS, No Category) and the OECD Test Guideline 439 standards, the exosome product described above is identified as non-irritating to the skin.

### Efficacy Tests of the Liquid Hair Growth Formulation.

After passing the safety tests, efficacy tests of the liquid hair growth formulation were conducted, including a human hair follicle cell proliferation assay, an assay on influence of hair growth regulation mechanisms, an animal model verification, and clinical cases.

### I. Human Hair Follicle Cell Proliferation Assay

In this assay, human follicle dermal papilla cells (HFDPCs) were used to evaluate whether the liquid hair growth formulation could promote the proliferation of human hair follicle cells.

In this assay, immortalized human follicle dermal papilla cells (HFDPCs, Abm) were used as test subjects. An experimental group was treated by adding the liquid hair growth formulation of the present invention into the culture medium. At the same time, two control groups were established: a blank control group containing only the cell culture medium, and a positive control group containing CHIR99021. The CHIR99021 is an inhibitor of glycogen synthase kinase-3 (GSK-3). GSK-3 is an important enzyme that primarily regulates various cellular functions, including metabolism, apoptosis, and growth, through phosphorylation. In hair follicle growth, GSK-3 plays a critical inhibitory role by degrading β-catenin protein in the Wnt/β-catenin signaling pathway. The Wnt/β-catenin pathway is a key driver of hair follicle development and hair regeneration. Therefore, by inhibiting GSK-3, CHIR99021 can activate the Wnt pathway and promote the proliferation of hair follicle cells, making it an effective positive control group for evaluating hair growth efficacy.

The culture method for immortalized HFDPCs involves seeding cells in a 96-well plate at a density of 4,500 cells/cm² and culturing overnight at a temperature of 37° C. and a CO2 concentration of 5% in a humidified environment. Subsequently, different concentrations of the liquid hair growth formulation or positive control substances are added to the culture medium, and the cells are cultured for an additional 72 hours. Finally, cell viability is assessed using the CellTiter-Glo® Luminescent Cell Viability Assay kit. The assay results are shown in Table 7.

TABLE 7
Results of human hair follicle cell proliferation assay, wherein
*p-value < 0.05 indicates a statistically significant
difference between the experimental and control groups.

| | | | Cell viability (%) |
|---|---|---|---|
| | | | (Average ± |
| | Additive in | | Standard |
| | culture | | Deviation, |
| Group | medium | Concentration | n = 3) |
| Experimental | Liquid hair | 0.156% | 97.7 ± 4.9 |
| group | growth | 0.313% | 100.5 ± 2.6 |
| | formulation | 0.625% | *110.6 ± 4.1 |
| | (vol %) | 1.25% | *114.3 ± 2.9 |
| | | 2.5% | *115.6 ± 1.9 |
| | | 5% | *136.2 ± 5.6 |
| | | 10% | *142.4 ± 4.0 |
| | | 20% | *195.6 ± 4.1 |
| Positive control | CHIR99021 | 1 μM | *114.0 ± 3.4 |
| group | | | |
| Blank control group | None | None | 100.0 ± 1.4 |

As shown in Table 7, the liquid hair growth formulation promoted the proliferation of human hair follicle cells in a dose-dependent manner, increasing with the amount of formulation added. Notably, when the concentration of the liquid hair growth formulation reached 20%, the cell viability remarkably increased to 195.6±4.1%.

### II. Actual Cases of Voluntary Subjects.

In this experiment, the efficacy and safety of exosomes derived from DPCs as a non-invasive liquid hair growth formulation on humans will be evaluated. Evaluation criteria emphasize not only visible hair growth but also molecular-level indicators of hair follicle health, particularly changes in telomere length.

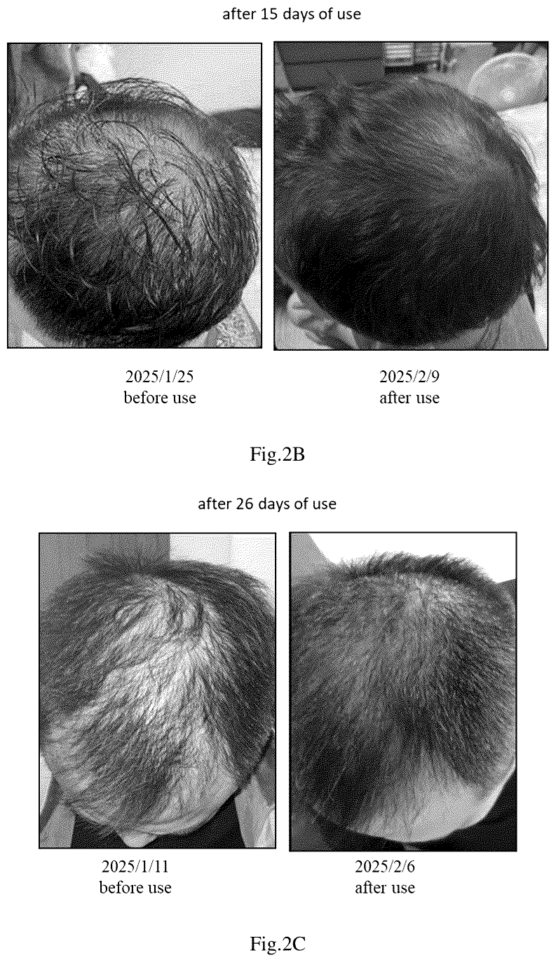

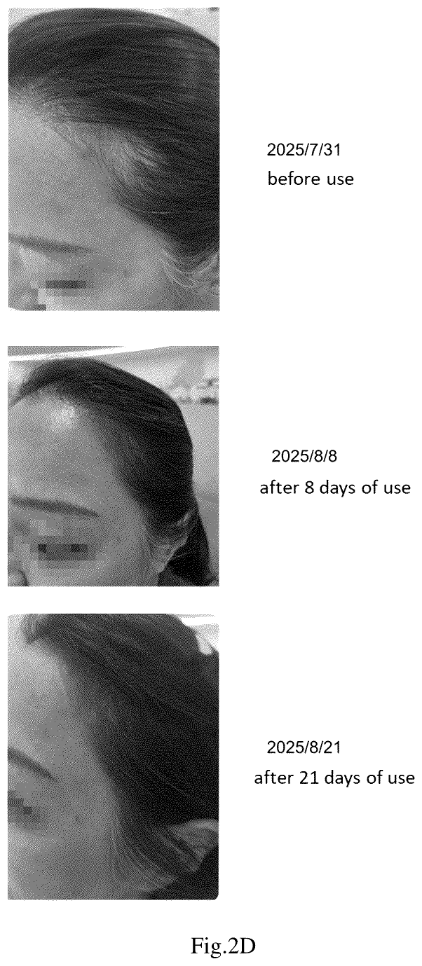

First, several volunteer subjects suffering from androgenetic alopecia were recruited for experiments. The experiments were designed as a non-blinded study, in which hair conditions of the subjects were evaluated both before treatment and after a period of product use. A liquid hair growth formulation containing exosomes derived from human dermal papilla stem cells at a concentration of ≥10³/mL was topically sprayed onto hair loss sites (target hair growth sites) of scalp of the volunteer subjects. After spraying the liquid hair growth formulation, the scalp was gently massaged to facilitate penetration of the formulation into the scalp of the volunteer subjects. FIG. 2A to 2D illustrate the comparative conditions of four volunteer subjects before and after using the liquid hair growth formulation. Although a usage period of the subjects shown in FIG. 2A to 2D varied from 14 to 26 days (<1 month), an increase in hair density and an expansion in a coverage area of hair were observed in the hair loss regions (i.e., target hair growth sites).

Additionally, the telomere length of hair follicle cells from the voluntary subjects was measured, and measurement results are shown in Table 8. According to the results in Table 8, after using the liquid hair growth formulation for 4 weeks, the telomere length of the hair follicle cells in the voluntary subjects was significantly increased. Since telomere shortening is an important marker of cellular aging, the increase in telomere length indicates that the liquid hair growth formulation not only promotes hair growth but also improves the health and vitality of hair follicle cells, fundamentally delaying an aging process of hair follicles and thereby providing a novel and effective strategy for the treatment of Androgenetic Alopecia.

TABLE 8
Measurement results of telomere length in hair follicle cells
after 4 weeks of using the liquid hair growth formulation.

| | Telomere Length Before | Telomere Length After Use |
|---|---|---|
| Test Case | Use (KB) | (KB) |
| Test 1 | 5.7 | 7.5 |
| Test 2 | 5.9 | 6.3 |

Furthermore, post-treatment self-assessments were obtained from 500 volunteer subjects, and results from a randomly selected subset of 50 are presented in Tables 9 and 10. As shown in Tables 9 and 10, by Week 2 (Day 14) of using the liquid hair growth formulation, volunteers already perceived noticeable improvements in hair growth, hair density, and scalp comfort, and similar perceptions were observed after 12 weeks of continued use. No adverse effects occurred throughout entire period of use (Weeks 2-12), further substantiating positive effects of the liquid hair growth formulation on hair follicle health and hair regrowth.

TABLE 9
Week 2 Post-Treatment Self-Assessment Results.

| | Strongly agree | Agree | Disagree |
|---|---|---|---|
| Hair growth | 38(76%) | 12(24%) | 0 |
| Hair density | 35(70%) | 15(30%) | 0 |
| Scalp comfort | 50(100%) | 0 | 0 |
| No adverse events (e.g., | 50(100%) | 0 | 0 |
| scalp irritation or | | | |
| tenderness) | | | |

TABLE 10
Week 12 Post-Treatment Self-Assessment Results.

| | Strongly agree | Agree | Disagree |
|---|---|---|---|
| Hair growth | 30(60%) | 20(40%) | 0 |
| Hair density | 40(80%) | 10(20%) | 0 |
| Scalp comfort | 32(64%) | 18(36%) | 0 |
| No adverse events | 50(100%) | 0 | 0 |
| (e.g., scalp irritation or | | | |
| tenderness) | | | |

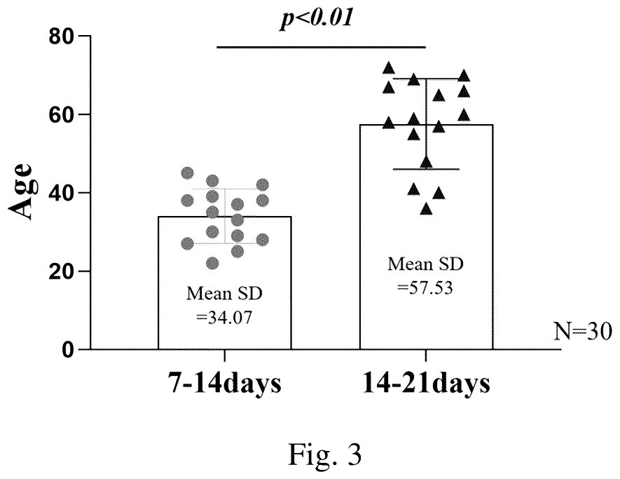

Finally, an analysis of the relationship between age and time to hair regrowth was conducted for these 50 volunteers; however, because several volunteers shared same age, and to ensure statistical rigor, only one subject per identical age band was retained, resulting in N=30. The results are shown in FIG. 3. As seen in FIG. 3, volunteers with a 7-14-day regrowth time had a mean age of 34.07, whereas those with a 14-21-day regrowth time had a mean age of 57.53. These findings indicate a positive correlation between hair-regrowth time and subject age.

The above shows that the present invention provides a novel and efficient liquid hair growth formulation, with the core advantages demonstrated in the following points:

Unique and controllable cell source: The present invention establishes a rigorous screening standard for super donors to select and isolate high-quality dermal papilla cells (DPCs) from occipital hair follicles of donors meeting specific age (35-45 years) and health conditions. This strategy ensures high quality and uniformity of the upstream cell source, providing a solid foundation for the stable production and high activity of subsequent exosomes. This unique cell screening process is innovative and maximizes the hair growth potential of exosomes.

High-purity and high-activity targeted exosomes: The preparation method of the present invention combines precise techniques such as immunomagnetic bead separation (MACS MicroBead) and tangential flow filtration (TFF) system. This not only effectively purifies and concentrates the exosomes but also ensures that the exosomes possess high concentrations of core markers (CD9, CD63, CD81). In vitro experiments have confirmed that these targeted exosomes can significantly promote the proliferation of human hair follicle cells in a dose-dependent manner, demonstrating outstanding biological activity.

Excellent biocompatibility and safety: According to an internationally recognized OECD TG 439 in vitro skin irritation test, the liquid hair growth formulation of the present invention shows no irritation to the reconstructed human epidermis model, thereby providing safety assurance for its application to the scalp. Furthermore, results from anti-inflammatory assays demonstrated that even at high concentrations, the exosomes of the present invention exhibited no evident cytotoxicity and did not induce inflammatory responses, further confirming their excellent biocompatibility.

Significant clinical efficacy and deep mechanism: The formulation of the present invention has been validated by the Franz diffusion cell system, demonstrating its ability to effectively penetrate the skin within a short time, ensuring that exosomes reach the target hair follicle area. Practical cases with voluntary subjects not only visually confirmed increased hair density but also first time proved its unique efficacy at the molecular level: significantly extending the telomere length of hair follicle cells. Since telomere length is an important indicator of cellular aging and health, this finding suggests that the formulation of the present invention not only promotes hair growth but also fundamentally improves the vitality of hair follicle cells, achieving a deep “rejuvenation” of hair follicles and providing unique advantages beyond traditional hair growth products.

The present invention has been disclosed herein by way of exemplary embodiments. However, it will be understood by those skilled in the art that these embodiments are provided for illustrative purposes only and are not intended to limit the scope of the claimed invention. Any modifications or substitutions that are equivalent or have a substantially equivalent effect to the embodiments described above should be interpreted as falling within the spirit or scope of the present invention. Accordingly, the scope of protection for the present invention shall be defined by the following claims.

What is claimed is:

1. A method for preparing targeted dermal papilla cell-derived exosomes, comprising:
isolating a plurality of dermal papilla cells expressing a cell marker CD133 (hereinafter referred to as CD133+ DPCs);
culturing the CD133+ DPCs in a two-dimensional culture way for primary culture, thereby obtaining a plurality of cell masses by growth of the CD133+ DPCs;
subculturing the cell masses to obtain a plurality of spindle-shaped cell lines exhibiting adherent growth;
collecting a cell culture medium containing a plurality of exosomes present in a surrounding environment during growth of the cell lines; and
isolating the exosomes from the cell culture medium to obtain an exosome solution, wherein the exosomes express surface markers of CD9, CD63, and CD81.

2. The method of claim 1, wherein the dermal papilla cells are obtained from occipital hair follicles of a healthy human donor in compliance with local health regulations and regardless of gender.

3. The method of claim 1, wherein the conditions of the primary culture and the subculture comprise being carried out in a laboratory of a GMP cell preparation facility, being conducted under a xeno-free and serum-free culture environment, and maintaining a temperature of 37° C. and a carbon dioxide concentration of 5%.

4. The method of claim 1, wherein the methods for isolating the CD133+ DPCs comprise an immunomagnetic bead separation method.

5. The method of claim 1, wherein the methods for isolating the exosomes comprise a tangential flow filtration system, a size exclusion chromatography, or a combination thereof.

6. The method of claim 1, wherein the composition of the exosome solution comprises:
the exosomes at a concentration of 3.5×10⁸/mL to 4.5×10¹⁴/mL, wherein the exosomes have a particle size of 30 to 160 nm; and
proteins at a concentration of 3000 to 3200 mg/mL.

7. An exosome product, wherein a method for preparing the exosome product comprising:
obtaining the exosome solution by using the method for preparing targeted exosome as claimed in claim 1; and
lyophilizing the exosome solution to obtain the exosome product in a lyophilized powder form,
wherein a positivity rate for a surface marker CD9 is greater than 20%, for a surface marker CD63 is greater than 18%, and for a surface marker CD81 is greater than 7%.

8. A liquid hair growth formulation, wherein the formulation prepared by dispersing the exosome product as claimed in claim 7 in a solvent;
wherein the formulation comprises the exosomes at a concentration of 10³ to 10⁸/mL.

9. The formulation of claim 8, wherein the solvent of the formulation is normal saline or a solution comprising other components in liquid form.

10. A method of using the liquid hair growth formulation as claimed in claim 8, comprising:
spraying the formulation onto a target hair growth site on the scalp of an individual; and
massaging the target hair growth site of the individual to promote penetration of the exosomes into a plurality of hair follicles of the scalp, thereby stimulating the hair follicles to grow hair.
